(19) United States
(12) Patent Application Publication
(10) Pub. No.: US 2007/0141561 A1
(43) Pub. Date: Jun. 21, 2007

(54) MICROFLUIDIC DEVICE AND METHOD FOR CONCENTRATION OR PURIFICATION OF SAMPLE CONTAINING CELLS OR VIRUSES

(75) Inventors: Su hyeon KIM, Seoul (KR); Yoon kyoung CHO, Suwon-si (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD.,, Suwon-si (KR)

(21) Appl. No.: 11/555,763

(22) Filed: Nov. 2, 2006

(30) Foreign Application Priority Data
Dec. 20, 2005 (KR) 1020050126262

(51) Int. Cl. C12Q 1/70; C12N 7/02

(52) U.S. Cl. 435/5; 204/547; 435/239

(57) ABSTRACT

A microfluidic device for concentrating or purifying a sample containing cells or viruses including a sample circulating vessel having a constant capacity; a pump connected to the sample circulating vessel and a trapping unit located in the sample circulating vessel. A method of concentration or purifying a sample containing cells or viruses including introducing the sample containing cells or viruses into a sample circulating vessel, circulating the sample within the sample circulating vessel by a pump connected to the sample circulating vessel, capturing the cells or viruses contained in the sample at a specific site inside the sample circulating vessel to concentrate or purify the sample and recovering the concentrated or purified sample from the sample circulating vessel.

This application claims priority to Korean Patent Application No. 10-2005-0126262, filed on Dec. 20, 2005, and all the benefits accruing therefrom under 35 U.S.C. §119, the contents of which are incorporated herein in its entirety by reference.

### BACKGROUND OF THE INVENTION

1. Field of the Invention

The present invention relates to a microfluidic device for concentration and purification of a sample containing cells or viruses, and a method of concentrating or purifying a sample containing cells or viruses using the microfluidic device.

2. Description of the Related Art

Biological analysis processes, such as detection of pathogenic bacteria and molecular diagnosis, require sample pretreatment in order to enhance the detection sensitivity.

Sample pretreatment includes processes of eliminating any material which interrupts analysis through concentration, purification or the like, and of increasing the sample concentration to facilitate the analysis.

During such processes of pre-treating a sample containing biological particles, the biological particles can be captured using dielectrophoretic force, electromagnetic force, optical force and the like, for the purpose of performing concentration and purification of the sample. However, known particle-capturing methods exhibit low efficiencies for capturing biological particles. Furthermore, in order to predict the quantity of the initial sample from the results obtained by analyzing a concentrated sample, a separate means or process for calculating the total volume of the initial sample is required, thus making automation of the pretreatment process difficult. Thus, attempts have been made, and are still being continued, to maximize the concentration and purification efficiency of the sample treatment process.

### BRIEF SUMMARY OF THE INVENTION

An exemplary embodiment provides a microfluidic device for efficiently concentrating or purifying a sample containing cells or viruses.

An exemplary embodiment provides a method of concentrating or purifying a sample containing cells or viruses using the microfluidic device.

In an exemplary embodiment, there is provided a microfluidic device for concentration or purification of a sample containing cells or viruses including a sample circulating vessel having a constant capacity, a pump connected to the sample circulating vessel and a trapping unit disposed inside the sample circulating vessel.

In an exemplary embodiment, there is provided a method of concentrating or purifying a sample containing cells or viruses using the microfluidic device. The method includes introducing the sample containing cells or viruses into the sample circulating vessel, circulating the sample within the sample circulating vessel by the pump connected to the sample circulating vessel, capturing the cells or viruses contained in the sample at a specific site inside the sample circulating vessel to concentrate or purify the sample and recovering the concentrated or purified sample from the sample circulating vessel.

An exemplary embodiment of the microfluidic device and method of the present invention are useful for enhancing the efficiency of concentration or purification of a sample containing cells or viruses.

### BRIEF DESCRIPTION OF THE DRAWINGS

The above and other features and advantages of the present invention will become more apparent by describing in detail exemplary embodiments thereof with reference to the attached drawings in which:

FIG. 1A, 1B and FIG. 2 are schematic diagrams illustrating the operating principle of an exemplary embodiment of a microfluidic device according to the present invention;

FIG. 3A and FIG. 3B are an exploded view and a perspective view, respectively, of an exemplary embodiment of a trapping unit according to the present invention;

FIG. 4A and FIG. 4B are a planar view and a lateral view, respectively, of a structure of an exemplary embodiment of an electrode portion arranged in the form of an array of flat metal plates and installed in a trapping unit of a microfluidic device according the present invention;

FIG. 5 and FIG. 6 are diagrams illustrating exemplary embodiments of dimensions of the electrode portion installed in the trapping unit according to the present invention;

FIG. 7 is a diagram illustrating an exemplary embodiment of dimensions of the trapping unit according to the present invention;

FIG. 8 is a graph showing the cell concentration efficiency according to time for a sample circulating inside an exemplary embodiment of a sample circulating vessel, at flow rates of 20, 60, 100 and 250 μl/min; and

FIG. 9 is a graph showing the cell concentration efficiency, expressed as a ratio of cell flow rate/rotation speed, for a sample circulating inside an exemplary embodiment of a sample circulating vessel, at flow rates of 20, 60, 100 and 250 μl/min.

### DETAILED DESCRIPTION OF THE INVENTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the present invention will be described in detail by explaining embodiments of the invention with reference to the attached drawings.

In an exemplary embodiment of the present invention, there is provided a microfluidic device for concentration or purification of a sample containing cells or viruses.

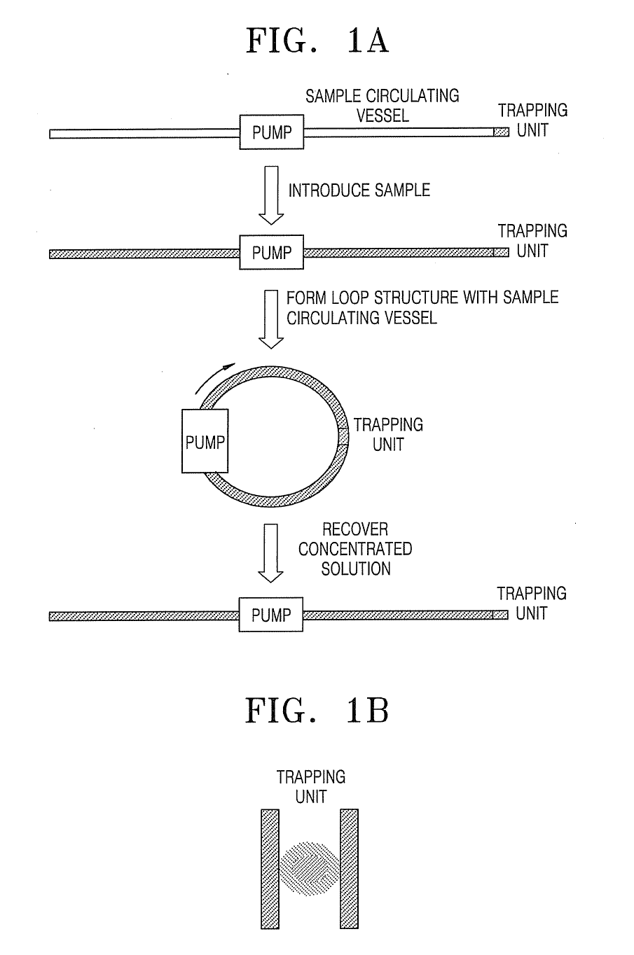

FIG. 1 and FIG. 2 are schematic diagrams illustrating the operating principle of an exemplary embodiment of a microfluidic device according to the present invention.

Referring to FIG. 1, the microfluidic device includes a sample circulating vessel having a substantially constant capacity, a pump connected to the sample circulating vessel and a trapping unit disposed inside the sample circulating vessel.

The sample circulating vessel includes a substantially constant capacity. Any of a number of vessels can be used as the sample circulating vessel, provided that the total volume of the sample introduced into the vessel can be precisely predicted. When the total volume of the sample introduced initially to the sample circulating vessel is known, the initial amount of the material to be analyzed in the sample can be calculated from the concentration efficiency of biological particles in the sample, which have been captured by the trapping unit. If the capacity of the sample circulating vessel were variable, it would be necessary to add a metering pump or a separate element for quantification in order to measure the initial sample volume.

The sample circulating vessel is required to be in a linear or bended form initially, but to have a loop-type structure when the sample circulating vessel is filled with the sample (See, FIG. 2A). Exemplary embodiments of such a sample circulating vessel include tubes and the like, but are not limited thereto. When a tube is used as the sample circulating vessel, processes of introducing the sample and discharging the treated sample can be performed relatively easily, thus making a tube advantageous for an automated system, and the initial sample volume can be easily determined from the inner diameter and length of the tube. In an exemplary embodiment when a tube is used as the sample circulating vessel, a flexible tube may be used so that a loop can be formed more easily.

Exemplary embodiments of the pump that can be used for the microfluidic device include a peristaltic pump and a membrane pump, but are not limited thereto. In an exemplary embodiment, the pump is connected to the sample circulating vessel so that the sample introduced into the sample circulating vessel can be circulated within the sample circulating vessel. The pump can be also installed directly on a specific position of the sample circulating vessel.

In an exemplary embodiment, the pump used for the microfluidic device should be of a type which is not brought into direct contact with the sample inside the sample circulating vessel, such as to prevent contamination of the pump. In one exemplary embodiment, a peristaltic pump may be used for the microfluidic device.

As in the illustrated exemplary embodiment, the trapping unit of the microfluidic device is disposed in the sample circulating vessel. This trapping unit may include a dielectrically separated crossing electrode portion which is capable of generating an electric field, a magnet which is capable of generating magnetic force, or an optical tweezer which are capable of generating optical force. However, the present invention is not limited thereto, and any of a number of methods and/or fixtures capable of capturing cells or viruses in the sample can be used.

In an exemplary, the trapping unit may be operated by dielectrophoresis. In this case, the trapping unit includes a dielectrically separated crossing electrode portion which is capable of generating an electric field. The trapping unit may include an upper substrate and a lower substrate and/or the crossing electrode portion may be located on the lower substrate of the trapping unit.

In exemplary embodiments, the crossing electrode portion may include metal plate columns, each of the metal plate columns consisting of two or more rows of metal plates arranged substantially perpendicularly to the direction of the sample flow. Among the metal plate columns, odd-numbered columns may be connected to a metal pad through metal wires, while even-numbered columns may be connected to another metal pad through metal wires.

The metal plates of the odd-numbered metal plate columns may be arranged alternately with the metal plates of the even-numbered metal plate columns.

The metal plates, metal pads and metal wires may be formed from any of a number of metals, such as a metal selected from the group consisting of gold and platinum. The metal is a biocompatible metal which is suitable for use with cells, such as gold. In an exemplary embodiment, the metal plates, metal pads and metal wires can be produced using a micropatterning technique.

In one exemplary embodiment, the interval (e.g. the distance between a gold plate on one column and a gold plate on another column) between a metal plate of an odd-numbered metal plate column and a metal plate of an even-numbered metal plate column may be about 10 microns (μm) to about 100 microns (μm).

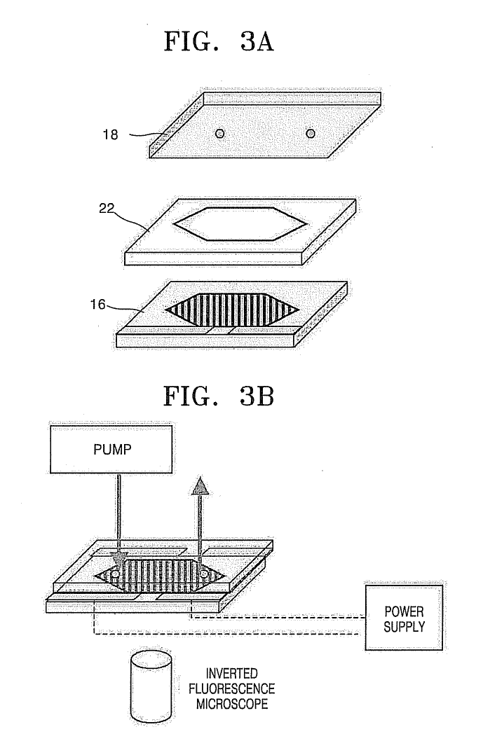

FIG. 4A and FIG. 4B are a planar view and a lateral view, respectively, of metal plates 20 and 20′, metal pads 10 and 10′ and metal wires 12 and 12′ of an exemplary embodiment of the crossing electrode portion of the trapping unit of the microfluidic device.

FIG. 4A illustrates metal plates 20 of even-numbered columns and metal plates 20′ of odd-numbered columns that are arranged substantially perpendicularly to the direction of the sample flow. In the illustrated exemplary embodiment, each column consists of four metal plates 20 and 20′, respectively. The metal plates 20′ of the odd-numbered columns are connected to one of the metal pads 10′ through metal wires 12′, while the metal plates 20 of the even-numbered columns are connected to another of the metal pads 10 through metal wires 12. As illustrated in FIG. 4B, the trapping unit includes an upper substrate 18 and a lower substrate 16. The metal plates 20 and 20′ are alternately disposed on the lower substrate 16 of the trapping unit.

In an alternative exemplary, the trapping unit may be driven by magnetic force, and thus, the trapping unit employs a magnet. In this case, magnetic particles having a size ranging from a few nanometers to a few micrometers are used. These magnetic particles may be coated with antibodies or nucleic acids on the surface thereof, so that the magnetic particles can selectively bind to the biological particles (e.g., cells or viruses) contained in the sample and can be collected using a strong magnet.

In an exemplary embodiment, the magnetic particles that are used to capture the cells or viruses are used for the sample concentration and purification in a batch mode. In one exemplary embodiment, a sample solution prepared by mixing a sample containing cells or viruses and magnetic particles, is introduced to the sample circulating vessel while not operating the trapping unit. The sample solution is then circulated while applying magnetic force to capture the cells or viruses in the sample solution. Controlling the magnetic force applied to the trapping unit may include, but is not limited to, a method of using a permanent magnet and adjusting the distance between the permanent magnet and the trapping unit, a method of using an electromagnet and controlling the intensity of the current applied to the electromagnet,; and the like.

In an alternative exemplary embodiment, it is possible to use a method of placing magnetic particles in the sample circulating vessel in advance, introducing the sample solution into the sample circulating vessel while applying magnetic force, circulating the sample solution in the absence of the magnetic force to mix the magnetic particles and the sample solution, and then circulating the sample solution while applying the magnetic force again to capture biological particles in the sample solution. In this embodiment, a mixer element may be included in the sample circulating vessel to help mixing of the sample solution.

In an alternative exemplary, the trapping unit may be driven by optical force. The trapping unit includes an optical tweezer. The optical force is created by the momentum transfer from photons to the biological particles, as well as the gradient force generated inside the biological particles in the sample. Equilibrium between the two effects enables capturing of the biological particles. Optical tweezers may be disadvantageous in that the region of capture is so small that it is difficult to capture a large number of biological particles. To allow a large number of biological particles to be captured, the optical tweezers may be scanned or a number of optical tweezers may be created in the space through holography.

In an exemplary embodiment, a method of concentrating or purifying a sample containing cells or viruses using the microfluidic device includes a) introducing a sample containing cells or viruses to the sample circulating vessel, b) circulating the sample within the sample circulating vessel by the pump connected to the sample circulating vessel, c) capturing the cells or viruses in the sample at a specific site inside the sample circulating vessel to concentrate or purify the sample and d) recovering the concentrated or purified sample from the sample circulating vessel.

Referring to FIGS. 2A and 2B, a sample containing cells or viruses is first introduced into the sample circulating vessel.

Subsequently, when the sample fills the sample circulating vessel, the two ends of the sample circulating vessel are connected to form a loop structure (FIG. 2C), and then a pump connected to the sample circulating vessel is operated to circulate the sample within the sample circulating vessel (FIG. 2D).

While the sample is circulated in the sample circulating vessel, the sample is concentrated or purified by capturing the cells or viruses in the sample at a specific site inside the sample circulating vessel. During this process, a method of generating a non-uniform electric field by a dielectrically separated crossing electrode portion, a method of using magnetic force created by a magnet, or a method of using optical force created by an optical tweezer can be used to capture the cells or viruses.

In an exemplary embodiment, the process of concentrating or purifying the sample may be performed by applying a voltage to the crossing electrode portion disposed in the sample circulating vessel and creating a spatially non-uniform electric field in a chamber of the sample circulating vessel, thereby capturing the cells or viruses in the sample. The applied voltage may be a direct current (DC) voltage or an alternating current (AC) voltage. The direct current voltage may have a magnitude ranging from about 1 volt (V) to about 100 volts (V). The alternating current voltage may have a magnitude ranging from about 1 V to about 100 V and a frequency of 100 Hz to 100 MHz. When a spatially non-uniform electric field is created inside the sample circulating vessel, the cells or viruses in the sample are captured at the crossing electrode portion by dielectrophoresis and eventually concentrated.

In an exemplary embodiment, the process of concentrating or purifying the sample may be performed by capturing the cells or viruses in the sample using a magnet disposed at a specific site inside the sample circulating vessel.

In an exemplary embodiment, the process of concentrating or purifying the sample may be performed by capturing the cells or viruses in the sample using a laser beam irradiated from an optical tweezers disposed at a specific site inside the sample circulating vessel.

When the sample is sufficiently circulated in the sample circulating vessel, the sample passes many times over the trapping unit located inside the sample circulating vessel, thereby achieving a desired concentration efficiency. The concentrated or purified sample is recovered from the sample circulating vessel to achieve concentration or purification of the cells or viruses contained in the sample (FIGS. 2E and 2F).

In exemplary embodiments, the sample containing cells or viruses may be selected from the group consisting of saliva, urine, blood, serum, and cell culture solution. The sample may contain animal cells, plant cells, bacteria, viruses, bacteriophages, or the like.

In an exemplary embodiment, the sample circulating vessel may be a tube and the tube may be a flexible tube.

In an exemplary embodiment, the pump may be a peristaltic pump or a membrane pump.

Hereinafter, the present invention will be described in detail with reference to Examples. The Examples are for illustrative purposes only, and are not intended to limit the scope of the present invention.

### EXAMPLE 1

### Production of Device According to the Present Invention

Tube: Tygon tube (Tygon® STR-3603/R-3607 (SC0002), Ismatec SA; inner diameter 0.25 millimeters (mm), length 33 mm, volume of solution in the tube: 16 μl);

Pump: Peristaltic pump (ISM596A, Ismatec SA, Switzerland);

The trapping unit was produced as follows.

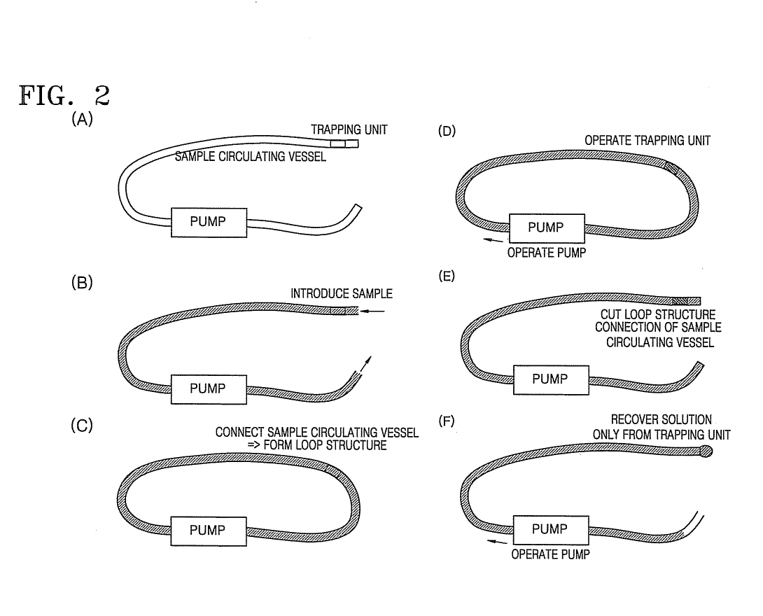

FIG. 3A and FIG. 3B illustrate an exploded view and a perspective view, respectively, of an exemplary embodiment of the trapping unit used in Examples 1 and 2. As illustrated in FIG. 3A, the trapping unit was produced by adhering an upper substrate 18 to a lower substrate 16 having an array of planar gold plates formed thereon, by using 3M™ adhesive tape 22 (3M Corporation, US). The upper substrate 18 and the lower substrate 16 were both made of glass.

Referring to FIG. 3B, a power supply is connected to the trapping unit via metal pads and an inverted fluorescence microscope (Nikon TE300) equipped with a CCD camera (Quantix 57, Roper Scientific, Inc.) is installed at the lower part of the trapping unit.

FIG. 4A and FIG. 4B illustrate the structure of the electrode portion of the trapping unit illustrated in FIG. 3A and FIG. 3B. FIG. 4A is a planar view of the electrode portion, which shows that four metal plate columns, each column consisting of four gold plate electrodes 20 and 20′, are respectively connected to gold pads 10 and 10′ through gold wires 12 and 12′. Here, the gold plates 20′ of odd-numbered columns are connected to one of the gold pads 10′, while the gold plates 20 of even-numbered columns are connected to another of the gold pads 10. FIG. 4B is a lateral view of the electrode portion, which shows that the gold plates are formed on the lower substrate 16. The metal plates 20 and 20′ alternate across a surface of the lower substrate.

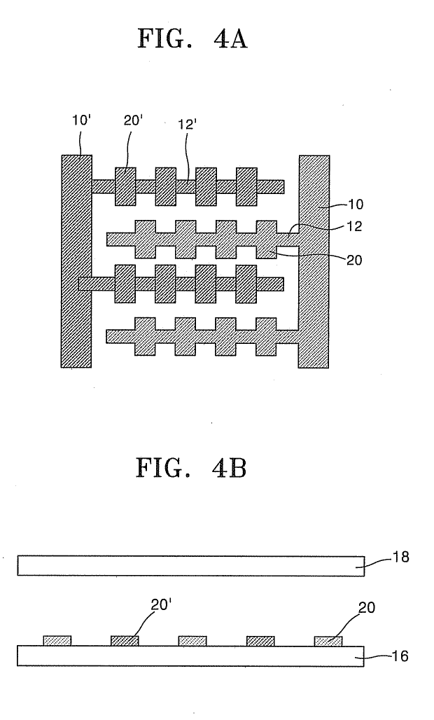

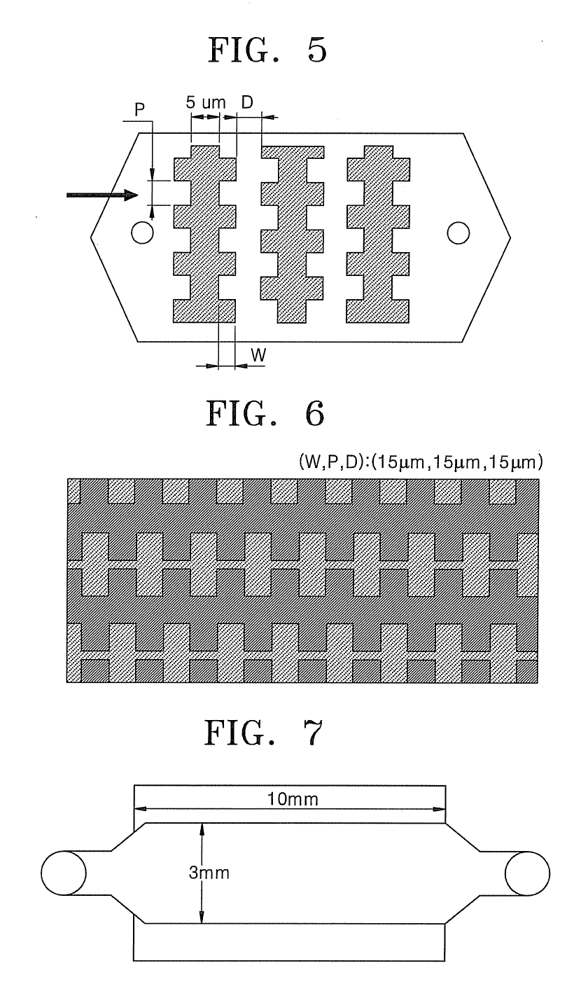

FIG. 5 and FIG. 6 are diagrams illustrating exemplary embodiments of dimensions of the electrode portion used in Example 1 and Example 2. Referring to FIG. 5, the width of the gold wire connecting the gold plates to the gold pad is 5 μm, the distance between a gold plate and another gold plate in the same column of gold plates is P (pitch), the width of a portion of a gold plate extending from the metal wire is W (width), and the distance between a gold plate on one column and a gold plate on another column is D. The arrow indicates the direction of the sample flow. The dimensions of the electrode portion produced in Example 1 were (W, P, D):(15 μm, 15 μm, 15 μm) (See FIG. 6). FIG. 7 is a diagram illustrating an exemplary embodiment of dimensions of the trapping unit used in Example 1. Referring to FIG. 7, the trapping unit used in Example 1 was 10 mm in width and 3 mm in length. The capacity of the trapping unit chamber was about 4 μl.

The tube was mounted on a peristaltic pump, and one end of the tube was connected to the inlet port of the trapping unit produced as described above. Then, the peristaltic pump was operated to introduce a cell solution through the outlet port of the trapping unit. When the tube and the trapping unit were completely filled with the cell solution without any foam, the other end of the tube was connected to the outlet port of the trapping unit to form a closed loop structure.

### EXAMPLE 2

### Concentration of Sample Containing Escherichia coli

In Example 2, a gram-negative bacterium, Escherichia coli (E. coli) (ATCC #11775), was cultured overnight in brain heart infusion (BHI) broth (Becton, Dickinson and Company, US) at 37° C. Subsequently, the culture solution was centrifuged at 5,000 rpm at 4° C. for 5 minutes, and then the cells were washed three times with a 0.1 mM sodium phosphate buffer solution. The conductivity was adjusted to 0.2 mS/m by diluting the cells with distilled water.

Then the E. coli cells were labelled with a cell staining solution (Live/Dead BacLight Bacterial Viability kit, Molecular Probe, Inc., US), and according to the product manual, 3 μl of a mixture of SYTO 9 and propidium iodide dye was added to 1 ml of the cell solution (OD₆₀₀=1) obtained above. In this manner, a solution of labelled E. coli cells in sodium phosphate buffer at a concentration of 10⁷ cells/ml was produced.

First, the tube was mounted on the peristaltic pump and one end of the tube was connected to the inlet port of the trapping unit produced in Example 1. Then, the 10⁷ cells/ml solution of labelled E. coli cells in sodium phosphate buffer was introduced through the outlet port of the trapping unit. When the cell solution completely filled the tube and the trapping unit up to the outlet port without any foam, the other end of the tube was connected to the outlet port of the trapping unit to form a closed loop structure.

Subsequently, the sample cell solution was circulated by the peristaltic pump at a flow rate of 20 μl/min. While the power supply was turned on to generate an electric field (20 V p-p, 100 kHz) in the trapping unit for 1 minute, fluorescence intensity images of the E. coli cells captured at the trapping unit were taken by the inverted fluorescence microscope equipped with a CCD camera. The images were analyzed with Metamorph (Universal Imaging Corporation).

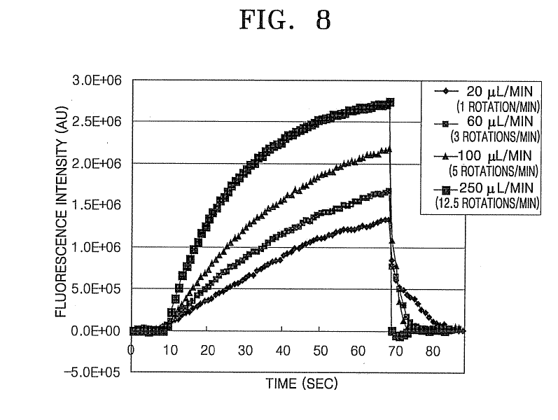

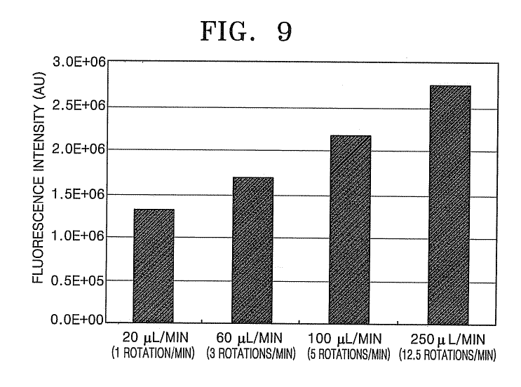

Meanwhile, the same procedure was performed while the flow rate of the cell solution circulating in the tube was changed to 60 μl/min, 100 μl/min and 250 μl/min, and the resulting fluorescence intensity images were analyzed. The results are presented in the following Table 1, as well as in FIG. 8 and FIG. 9. FIG. 8 is a graph showing the cell concentration efficiency according to time for the cell solution circulating inside the tube, at flow rates of 20, 60, 100 and 250 μl/min and FIG. 9 is a graph showing the cell concentration efficiency, expressed as a ratio of cell flow rate/rotation speed, for the cell solution circulating inside the tube, at flow rates of 20, 60, 100 and 250 μl/min.

TABLE 1

| Rotation speed | 1 Rotation/min | 3 Rotations/min | 5 Rotations/min | 12.5 Rotations/min |
|---|---|---|---|---|
| (Flow Rate) | (20 μl/min) | (60 μl/min) | (100 μl/min) | (250 μl/min) |
| 1 Rotation | 1.35E+06 (AU) | 8.77E+05 (AU) | 8.47E+05 (AU) | 8.10E+05 (AU) |
| 2 Rotations | | 1.40E+06 (AU) | 1.37E+06 (AU) | 1.29E+06 (AU) |
| 3 Rotations | | 1.68E+06 (AU) | 1.74E+06 (AU) | 1.63E+06 (AU) |
| 4 Rotations | | | 2.03E+06 (AU) | 1.92E+06 (AU) |
| 5 Rotations | | | 2.18E+06 (AU) | 2.12E+06 (AU) |
| 6 Rotations | | | | 2.29E+06 (AU) |
| 7 Rotations | | | | 2.42E+06 (AU) |
| 8 Rotations | | | | 2.53E+06 (AU) |
| 9 Rotations | | | | 2.60E+06 (AU) |
| 10 Rotations | | | | 2.67E+06 (AU) |
| 11 Rotations | | | | 2.71E+06 (AU) |
| 12 Rotations | | | | 2.75E+06 (AU) |

As illustrated in Table 1, a higher flow rate resulted in a decreased capturing efficiency per one circulation, but the overall capturing efficiency within the same time period increased with the flow rate. Specifically, when comparing a flow rate of 20 μl/min (1 circulation/min) and a flow rate of 250 μl/min (12.5 circulations/min), it can be seen that the fluorescence intensities after 1 circulation (1 minute taken) and 12 circulations (with a flow rate of 12.5 circulations per minute, 57.6 seconds taken for 12 circulations) were 1.35E+06 (AU) and 2.75E+06 (AU), respectively. Thus, the flow rate of 250 μl/min resulted in a capturing efficiency which was about two-folds higher than the capturing efficiency when the flow rate was 20 μl/min.

As an exemplary embodiment of the microfluidic device allows continuous circulation of the sample so that the sample can pass over the trapping unit many times while being circulated inside the sample circulating vessel at high flow rates, the concentration efficiency is enhanced. Since the sample circulating vessel has a substantially constant capacity, and an additional process of measuring the initial volume of the sample is not required, the amount of the analyzed material in the initial sample can be relatively simple calculated from the concentration efficiency measured as described above.

An exemplary embodiment of the microfluidic device employs a sample circulating vessel which maintains the sample at a constant volume and allows the sample to pass over the trapping unit many times, thus increasing the capturing efficiency. Advantageously, since the capacity of the sample circulating vessel is maintained constant, the initial volume of the sample can be obtained without any additional measurement thereof, and thus the amount of the analyzed material in the initial sample can be easily calculated.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A microfluidic device for concentrating or purifying cells or viruses, comprising:
a sample circulating vessel having a constant capacity;
a pump connected to the sample circulating vessel; and
a trapping unit disposed inside the sample circulating vessel.

2. The device of claim 1, wherein the trapping unit comprises a dielectrically separated crossing electrode portion that generates an electric field.

3. The device of claim 2, wherein the trapping unit further comprises an upper substrate and a lower substrate and the crossing electrode portion is formed on the lower substrate.

4. The device of claim 2, wherein the crossing electrode portion comprises: two or more rows of metal plate columns, each of the metal columns including an array or two or more metal plates arranged perpendicularly to a direction of a sample flow,
wherein the metal plates of odd-numbered columns are connected to a first metal pad through first metal wires and the metal plates of even-numbered columns are connected to a second metal pad through second metal wires.

5. The device of claim 4, wherein the metal plates of odd-numbered columns are arranged alternately with the metal plates of even-numbered columns.

6. The device of claim 4, wherein an interval between a metal plate of an odd-numbered column and a metal plate of an even-numbered column is about 10 to 100 μm.

7. The device of claim 5, wherein an interval between a metal plate of an odd-numbered column and a metal plate of an even-numbered column is about 10 to 100 μm.

8. The device of claim 1, wherein the trapping unit comprises a magnet.

9. The device of claim 1, wherein the trapping unit comprises an optical tweezer.

10. The device of claim 1, wherein the sample circulating vessel is a tube.

11. The device of claim 10, wherein the tube is a flexible tube.

12. The device of claim 1, wherein the pump is a membrane pump.

13. The device of claim 1, wherein the pump is a peristaltic pump.

14. A method of concentrating or purifying a sample containing cells or viruses, the method comprising:
introducing the sample containing cells or viruses into a sample circulating vessel, the sample circulating vessel having a constant capacity;
circulating the sample within the sample circulating vessel by a pump connected to the sample circulating vessel;
capturing the cells or viruses contained in the sample at a specific site inside the sample circulating vessel to concentrate or purify the sample; and
recovering the concentrated or purified sample from the sample circulating vessel.

15. The method of claim 14, wherein the capturing the cells or viruses includes applying a voltage to a dielectrically separated crossing electrode portion disposed at a specific site in the sample circulating vessel and generating a spatially non-uniform electric field in a chamber of the sample circulating vessel.

16. The method of claim 14, wherein the capturing the cells or viruses includes using a magnet disposed at a specific site in the sample circulating vessel.

17. The method of claim 14, wherein the capturing the cells or viruses includes using a laser beam irradiated from an optical tweezer disposed at a specific site in the sample circulating vessel.

18. The method of claim 14, wherein the sample circulating vessel is a tube.

19. The method of claim 18, wherein the tube is a flexible tube.

20. The method of claim 14, wherein the pump is a membrane pump.

21. The method of claim 14, wherein the pump is a peristaltic pump.
